(19) United States
(12) Patent Application Publication
(10) Pub. No.: US 2018/0349551 A1
(43) Pub. Date: Dec. 6, 2018

(54) SYSTEM AND METHOD FOR PATTERNING ANALYSIS OF A TELOMERE LENGTH DATASET

(71) Applicant: Timothy Frederick CARTER, Guelph (CA)

(72) Inventors: Timothy Frederick CARTER, Guelph (CA); Daniel Joseph GILLIS, Guelph (CA); William Allan KING, Guelph (CA)

(21) Appl. No.: 15/612,474

(22) Filed: Jun. 2, 2017

(51) Int. Cl. G06F 19/20; C12Q 1/68

(52) U.S. Cl. CPC G06F 19/20; C12Q 1/6841

(57) ABSTRACT

The present invention relates a system and method for analysis of telomere patterning. The method includes: capturing a dataset of telomere length using a measurement technique; determining component distributions of the dataset relating to telomere length; determining primary values between each of the component distributions, the primary values representing a pairwise assessment of a level of similarity between the component distributions; determining higher order values of at least one higher order assessment of each of the primary values; and outputting the primary values and the higher order values.

### TECHNICAL FIELD

The present invention relates to deoxyribonucleic acid (DNA) analysis; and more particularly, to patterning analysis of a telomere length dataset.

### BACKGROUND

Within each mammalian cell, the linear ends of every chromosome are capped by a telomere consisting of (TTAGGG)ₙ DNA repeats and associated protein elements. The number of repeats present, or length of each telomere, appears to be determined by the combined interaction of subtelomeric, genetic and epigenetic elements, physiological and environmental components and the proliferative history of the cell. As a result of these influences, chromosome arm specific telomere length varies within a general population, but also shows characteristics specific to each animal or individual. Further, by virtue of these roles and relationships, telomeres are considered critical cellular components and important biomarkers capable of providing insight into cellular and organism age and health.

The specific components and characteristics of telomeres vary between organisms in reasonable association with overall genetic and taxonomic relationships; however, at their core and regardless of species, telomeres can generally be considered to consist of repetitive DNA elements and specialized associated proteins.

During an epigenetic reprogramming procedure, such as cloning by somatic cell nuclear transfer (SCNT) or induced pluripotent stem cell (iPSC) generation, the nucleus of an existing cell is used to give rise to a new animal or population of pluripotent stem cells respectively. While it is generally accepted that mean telomere length is also increased during these procedures to a level comparable to their natural counterparts, it is unknown if the patterns of chromosome specific telomere lengths in the original donor cell are recapitulated in the reprogrammed cells.

By virtue of their terminal position, telomeres are incompletely replicated during normal DNA synthesis. They are also liable to repeat loss through oxidative damage and deletion. As a result, telomere length generally shortens with cellular proliferation and age. During early embryo development and within privileged cellular compartments, such as stem cells, a specialized enzyme capable of synthesizing de novo telomere repeats known as telomerase is expressed to lengthen the telomeres and counteract telomere shortening. The establishment of proper telomere lengths during development and their maintenance over time is critical to cellular and organism health. Within the cell, telomere length influences cellular behaviour and a minimum telomere length is a requirement for continued cellular proliferation. A single telomere shortening to a critical length is enough to signal the cell to enter senescence or commit apoptosis. This action protects against genomic instability and oncogenic progression by preventing chromosome fusions and limiting the possibility of accumulating a critical load of deleterious or pro-oncogenic mutations in a given cell lineage. Therefore, maintenance of telomere lengths is critical for cellular and organism health.

The same subtelomeric, genetic and epigenetic, physiological and environmental components that give rise to chromosome specific telomere lengths can also lead to unequal rates of shortening across different telomeres during the aging process. For example, the telomeres of the epigenetically repressed inactive X (Xi) chromosome have been shown to lose repeats at a significantly increased rate compared to the active X (Xa) in human females. The use of epigenetically reprogrammed cells to preserve genetic lineages through healthy clones, or in regenerative medicine to replace failed tissues and organs, requires telomere lengths and telomere programs capable of maintaining their long-term genetic and proliferative stability. Carryover of age related telomere changes or improper reprogramming of the telomere program at single chromosome arms could compromise this capacity. Under conventional telomere measurement techniques that merely measures mean telomere length, the above types of alterations to telomere length may not be apparent in mean telomere length measurements.

It is therefore an object of the present invention to provide a system and method for patterning analysis of a telomere length dataset in which the above disadvantages are obviated or mitigated and attainment of the desirable attributes is facilitated.

### SUMMARY

In an aspect there is provided a computer-implemented method for patterning analysis of a telomere length dataset executed on a processing unit, the processing unit comprising one or more processors, the method comprising: capturing a dataset of telomere length using a computer-assisted measurement technique; generating component distribution datasets via analysis of the telomere length dataset; generating datasets of primary values by performing pairwise assessments of levels of similarity between component distribution datasets; generating datasets of higher order values comprising at least one higher order assessment of primary value datasets; and outputting the datasets of primary values and the datasets of higher order values.

In another case, the primary values, the at least one higher order comparison values, or both, are weighted based on the assigned significance.

In yet another case, the measurement technique is quantitative fluorescence in situ hybridization (FISH) on metaphase preparations (mqFISH).

In a further case, the measurement technique includes at least ten metaphases per sample.

In yet another case, the measurement technique is normalized with a centromere reference probe by averaging two centromere measurements in each metaphase to normalize the raw telomere measurements of that metaphase.

In yet another case, the measurement technique is normalized for each telomere length measurement by converting the measurements into a proportion of the sum of all telomere lengths considered in each given metaphase and component distribution.

In yet another case, the measurement technique is normalized for each telomere length measurement by normalizing to the mean telomere length value per metaphase.

In yet another case, each of the component distributions comprise one of a telomere versus telomere (TVT) distribution, a pair versus pair (PVP) distribution, and an arm versus arm (AVA) distribution.

In yet another case, the primary values are determined by a non-parametric Anderson-Darling (AD) assessment.

In yet another case, the primary values are determined by a Kolmogorov-Smirnov (KS) assessment.

In yet another case, the higher order assessment is selected from a group consisting of: determining a full comparison between the primary values; determining a chromosome with the most telomere dissimilarity using the primary values; and determining a telomere with the most dissimilarity using the primary values.

In another aspect, there is provided a system for patterning analysis of a telomere length dataset, the system comprising one or more processors and a data storage device, the one or more processors configured to execute, or direct to be executed: a measurement module for capturing a dataset of telomere length using a measurement technique from an input device; a component module for generating component distribution datasets via analysis of the telomere length dataset; an evaluation module for generating datasets of primary values by performing pairwise assessments of levels of similarity between component distribution datasets, and for generating datasets of higher order values comprising at least one higher order assessment of primary value datasets; and an output module for outputting the datasets of primary values and the datasets of higher order values.

In a particular case, the evaluation module assigns a measure of significance to the primary values, the at least one higher order comparison values, or both.

In another case, the evaluation module weighs the primary values, the at least one higher order comparison values, or both, based on the assigned significance.

In yet another case, the measurement technique is quantitative fluorescence in situ hybridization (FISH) on metaphase preparations (mqFISH).

In yet another case, each of the component distributions comprise one of a telomere versus telomere (TVT) distribution, a pair versus pair (PVP) distribution, and an arm versus arm (AVA) distribution.

In yet another case, the primary values are determined by a non-parametric Anderson-Darling (AD) assessment.

In yet another case, the primary values are determined by a Kolmogorov-Smirnov (KS) assessment.

In yet another case, the higher order assessment is selected from a group consisting of: determining a full comparison between the primary values; determining a chromosome with the most telomere dissimilarity using the primary values; and determining a telomere with the most dissimilarity using the primary values.

These and other aspects are contemplated and described herein. The foregoing summary sets out representative aspects of systems and methods to assist skilled readers in understanding the following detailed description.

### DESCRIPTION OF THE DRAWINGS

An embodiment of the present invention will now be described by way of example only with reference to the accompanying drawings, in which:

FIG. 1 is an exemplary quantitative fluorescence in situ hybridization (qFISH) image;

FIG. 2 is an exemplary image showing a sample of XY human mqFISH metaphase with FITC telomere signals;

FIG. 3 is a block diagram showing a system for patterning analysis of a telomere length dataset, according to an embodiment;

FIG. 4 is a flow chart showing a method for patterning analysis of a telomere length dataset, according to an embodiment;

FIG. 5 is a diagram showing the method of FIG. 4;

FIG. 6 is a chart showing the raw telomere measurements according to an example of the method of FIG. 4;

FIG. 7 is a chart showing normalized telomere values according to the example of FIG. 6;

FIG. 8 is a chart showing mean and centromere normalized telomere values according to the example of FIG. 6;

FIGS. 9, 10 and 11 are charts showing telomere versus telomere (TVT) component comparison distributions of samples A, B, and C, respectively, according to the example of FIG. 6;

FIGS. 12, 13 and 14 are charts showing pair versus pair (PVP) component comparison distributions of samples A, B, and C, respectively, according to the example of FIG. 6;

FIGS. 15, 16 and 17 are charts showing arm versus arm (AVA) component comparison distributions of samples A, B, and C, respectively, according to the example of FIG. 6;

FIGS. 18, 19 and 20 are charts showing primary 1° values for the TVT component values, PVP component values, and AVA component values, respectively, according to the example of FIG. 6;

FIG. 21 is a chart showing primary 1° values aggregated by chromosome, according to the example of FIG. 6;

FIG. 22 is a chart showing a comparison of primary 1° values aggregated by type of comparison, according to the example of FIG. 6;

FIG. 23 is a chart showing a comparison of significant primary 1° values aggregated by type of comparison;

FIG. 24 is a chart showing a second order 2° comparison of primary 1° comparison values, according to the example of FIG. 6;

FIG. 25 is a chart showing a CDC comparison of telomere lengths between male samples and sample types;

FIG. 26 is a chart showing a CDC comparison of telomere lengths between female samples and sample types; and

FIG. 27 is a chart showing a CDC comparison and critical values for overall sample groups.

### DETAILED DESCRIPTION

Embodiments will now be described with reference to the figures. For simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Any module, unit, component, server, computer, computing device, mechanism, terminal or other device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Any application or module herein described may be implemented using computer readable/executable instructions that may be stored or otherwise held by such computer readable media and executed by the one or more processors.

The present invention relates to deoxyribonucleic acid (DNA) analysis; and more particularly, to patterning analysis of a telomere length dataset.

The specific components and characteristics of telomeres vary between organisms in reasonable association with overall genetic and taxonomic relationships; however, at their core and regardless of species, telomeres can generally be considered to consist of repetitive DNA elements and specialized associated proteins.

Across mammals, telomeric DNA is made up of multiple tandem DNA repeats with the sequence (TTAGGG)ₙ. Non-mammals also have similar telomere repeat sequences suggesting the importance of repeat characteristics, such as nucleotide sequence, repetitiveness and GC content, for telomere function. In humans, the length of the average telomere is typically reported to be between 8-20 kilobases at birth, depending partly on measurement methodology, with significant variation between individuals and in the relative length of telomeres between chromosomes and chromosome arms. These variations in telomere length at specific chromosome arms appear to be consistent in an individual and show some conserved patterns across larger populations. For example, the chromosome 17 p-arm telomere has been reported as generally short in humans. Additionally, the telomeres of the short, or p-arms, of acrocentric and small submetacentric chromosomes also tend to be shorter than those found on the longer, q-arms, of the same chromosomes, or even in comparison to the p- and q-arms of larger submetacentric and metacentric chromosomes.

At their termini, telomeres maintain a 3′, G-rich single stranded overhang varying in length from tens to hundreds of nucleotides. This feature is critical for telomere function and integrity; it interacts with the originating telomere strand and other telomeric components to form T-loop, D-loop and G-quadruplex higher order structures that play important roles in telomere structure and function.

The double stranded (TTAGGG)ₙ repeat motif is recognized by the homodimeric proteins TRF1 (Telomere Repeat Factor 1) and TRF2 (Telomere Repeat Factor 2). The single stranded section of the telomere is recognized and bound by the POT1 (Protection of Telomere 1) protein. RAP1 (Repressor/Activator Protein 1) and TPP1 (TINT1/PTOP/PIP1; a combination of the three names originally given to the same protein a combination of the three names given to the same protein) are two stabilizing proteins that interact with TRF2 and POT1 respectively. The TIN2 (TRF1- and TRF2-Interacting Nuclear Protein 2) protein in turn can interact with TPP1, as well as TRF1 and TRF2, which allows it to act as a bridge between the double strand associated TRF1 and TRF2 and the single strand associated TPP1+POT1. Together these 6 subunits (TRF1, TRF2, POT1, RAP1, TPP1 and TIN2) are referred to as the shelterin complex and represent the proteins most closely associated with the telomere. The shelterin complex acts to directly compact and stabilize the telomere structure to shelter the terminal end of the telomere from recognition as a DNA break and from degradation by nucleases. Shelterin also plays a role in the regulation of telomere length and acts as an important interface between telomeres and other proteins and their associated signalling networks within the cell.

Directly proximal to the telomere there is a considerably larger subtelomere containing degenerate (TTAGGG)ₙ sequences and other repetitive DNA elements that is quite variable between chromosomes and individuals. Differences in subtelomere structure and associated elements, particularly epigenetic marks, have been tied to changes in telomere length and are presumed to be at least partially responsible for the patterning of telomere length at specific chromosomes. The variable and repetitive nature of subtelomeres complicates their investigation and they remain comparatively poorly understood in comparison to other areas of the genome.

Telomeres are dynamic structures with the potential to both shorten and lengthen; however, maintenance of telomere length within a normal range is critically necessary for telomere integrity and function and, by extension, normal cellular proliferation and organism health. Although some telomere shortening is associated with the aging process, shortening beyond a critical minimum leads to telomere failure and the loss of cellular proliferative capacity or genome integrity. Abnormally long telomeres can also be disadvantageous, particularly in relation to oncogenic risk.

Limitations in semi-conservative DNA replication machinery, conventionally called the “End Replication Problem”, as well as the presence and need to maintain the 3′ single stranded overhang results in telomere shortening during replication. Measurement of the number of repeats lost solely through replication is complicated by strand specific processing and the challenge of capturing replication loss in isolation from other telomere dynamics during experiments; however, in vitro estimates and mechanistic investigations from human cells suggest the mean rate of end replication associated erosion generally lies between 30-100 base pairs per round of division. Other telomere characteristics, specifically their heterochromatic state, highly repetitive nature and associated proteins and secondary structures, may also lead to larger, but also more occasional, replication associated losses through stalled and aborted or otherwise unsuccessful replication through the telomeres.

Oxidative stress and DNA breaks also result in the loss of telomeric DNA and telomere shortening. Similar to replication, it appears telomere characteristics also pose additional challenges in relation to DNA damage and repair, which can result in telomere repeat loss and shortening. For example, the GGG sequence within the telomere repeat appears particularly susceptible to oxidative damage and telomere associated proteins and chromatin states down-regulate and modify the normal DNA damage response and repair pathways. Importantly, erosion and telomere disruption due to oxidative stress represents a presumptive mechanism by which smoking, obesity and other metabolic and environmental factors influence telomere length.

Upon shortening to a critical length, a telomere is no longer able to maintain its structure and associated integrity. Upon losing its integrity, the telomere is unable to hide the terminal end of the chromosome and it is recognized as a DNA break. In humans, an absolute minimum length of 77 nucleotides of true telomeric DNA has been reported. However, it is likely telomere failure and telomere induced senescence or apoptosis occurs at a range above this minimum and the threshold for a critical length or signalling response may also vary across species and cell types. As the critically short telomere is not actually a suitable candidate for conventional repair, with no matching broken DNA strand, DNA repair must not take place and is inhibited by remaining telomere-associated factors. This continued presence of an unrepaired telomere break ultimately signals the cell to enter senescence or commit apoptosis via the p53 pathway. Failure to properly respond to telomere failure by entering senescence or committing apoptosis can rapidly lead to genetic imbalance during subsequent cellular divisions via chromosome fusions and breakage bridge fusion cycles.

By this mechanism, the telomeres act as hard limits on cellular proliferation and also guard against genome instability from erosion of the terminal end of the chromosome into the genome, or through chromosome end fusions. From a broader perspective, the proliferative limit imposed by the telomeres can also be seen to protect genome integrity by minimizing the possibility of a given somatic cell lineage having sufficient opportunity to accumulate and perpetuate a critical load of deleterious or pro-oncogenic mutations. Importantly, as a single critical length telomere can be sufficient to bring about senescence, apoptosis or severe instability with continued proliferation, in some cases, the shortest telomere and chromosome specific telomere lengths may be more relevant than mean telomere length when assessing cellular populations.

In cells where increased or continual proliferation is required, such as stem cells, a specialized reverse transcriptase telomerase, known as telomerase, can counteract telomere shortening. Using an RNA template (TERC) and a catalytic subunit (TERT), telomerase elongates telomeres by synthesizing de novo TTAGGG repeats onto the 3′ terminal end. In mammals, this lengthening of the telomeres by telomerase is reported to occur in a number of stages between the S and M stages of the cell cycle. Telomerase access to the telomere, and therefore its lengthening activity, is controlled by a number of factors, including shelterin proteins and telomere conformation. As the number and proportion of shelterin proteins present, as well as telomere conformation, can vary from chromosome arm to chromosome arm and in relation to telomere length, a related variation in telomerase activity at each given chromosome arm can also be expected. There is also evidence telomerase only elongates a subset of telomeres during each round of cellular division in non-cancerous cells and may have a preference for shorter telomeres.

In humans, expression of TERT, and therefore telomerase mediated telomere elongation, is tightly controlled and absent in the majority of adult cells. Interestingly, a large number of TERT splice variants have also been identified, along with roles for telomerase outside of telomere lengthening. While these non-canonical roles for telomerase are still being understood, associations with mitochondria and pluripotency have been reported.

In rare cases, lengthening of telomeres and the continuation of cellular division is accomplished through an Alternative Lengthening of Telomeres (ALT) pathway. This is notably observed and associated with the approximately 10% of cancer samples that are found to be telomerase-null. The ALT mechanism of telomere lengthening itself is not completely understood, but involves recombination-based amplification. ALT positive cells are also characterized by extremely heterogeneous telomere lengths, the presence of extrachromosomal telomere repeat containing DNA, as well as degenerate telomere repeats, such as TCAGGG. The epigenetic state, specifically hypomethylation, of the telomere and subtelomere have also been associated with increased rates of recombination at the telomeres and the ALT phenotype. Notably, a recombination-based mechanism potentially similar to ALT also appears to be responsible for rapidly reprogramming telomere length in the first few cellular divisions of the embryo following fertilization.

The epigenome is closely intertwined with many aspects of telomere biology. The telomere and subtelomere are regarded as classic heterochromatic regions and are marked by repressive epigenetic features including trimethylated H3K9 and H4K20, hypoacetylated H3 and H4 and methylated DNA. Experimental alterations in the telomere and subtelomere epigenetic signature are associated with changes in telomere length through altered regulation of telomerase activity or ALT and in, extreme cases, loss of telomere integrity. Observational evidence of strong links between irregular chromatin markings and telomere dynamics in oncogenesis and other disease states further demonstrates the importance of epigenetic regulation of the telomere and a healthy telomere and cellular phenotype. Upon integrating these reports into a comprehensive model an epigenetic telomere length feedback loop emerges in which telomere shortening coincides with the loss of heterochromatic features from the telomere and subtelomere, localized promotion of telomere lengthening, followed by the reestablishment of heterochromatic marks and the repression of telomere lengthening. Importantly, this model and the above observations suggest a stable increase in heterochromatic factors or the presence stubborn heterochromatic features at a given telomere or subtelomere will ultimately result in a decrease of its telomere length.

Despite the presence of lengthening mechanisms, overall telomere length steadily decreases with age in nearly all tissues. This trend, along with their critical role in allowing cellular proliferation has led to the generally accepted hypothesis that telomere mediated senescence represents a major driving force of the aging process. In this regard, telomere length is also considered a biological marker, analogous to a countdown, with longer telomere lengths generally representing younger and healthier cells and shorter telomere lengths representing aged cells and exposure to unhealthy factors.

In addition, examination of twin and familial cohorts has reported mean telomere length to be a highly heritable trait. There is some variation between studies regarding the significance and magnitude of each parent's influence as well as the significance of some other factors; however, there is a clear consensus for overall heritability and considerable evidence for the existence of both maternal and paternal heritable factors. For both maternal and paternal inheritance genomic imprinting has been suggested as a potential mechanism. Maternally, the DKC1 gene, located on the X-chromosome and coding for a telomerase interacting protein, and mitochondrial DNA have also been suggested as a possible factors. Paternally, a strong correlation between offspring telomere length and paternal age is observed as well, leading to the suggestion age related increases in sperm telomere length are themselves inherited. Irrespective of mode of inheritance, SNP association studies have reported relationships between variants in telomere-associated factors, such as TERC, and telomere length and longevity. A number of studies have also shown evidence for the inheritance of telomere length at the chromosome arm specific level and suggest telomere near DNA factors as a mechanism.

Telomere erosion has been consistently demonstrated to limit the ability of cells to proliferate. Classically, the restriction posed by telomeres and telomere shortening on the proliferative potential of the cell has been referred to as the “Hayflick Limit”. Upon reaching this limit, cells have exhausted their telomeres to a critical point and will not divide further barring transformation, such as reactivation of telomerase or conversion to an ALT phenotype. While a direct link between telomere imposed restrictions on proliferation and subsequent pathology is difficult to demonstrate during normal in vivo aging, mutations disrupting telomere or telomerase function and abnormally short telomeres are associated with premature aging, organ failure and other disease states. Most notably in humans, mutations in the telomerase enzyme or critical telomere factors leading to decreases in telomere function are observed in aplastic anaemia, dyskeratosis congenita bone marrow failure syndromes and idiopathic pulmonary fibrosis phenotypes. Investigations focused on telomere lengths within stem cell compartments have also shown age related loss and correlation with the emergence of age related characteristics in tissues and organ systems. In animal models of human aging, such as telomerase null mice, the onset of very short telomeres coincides with impaired organ regeneration, earlier onset of functional age-related deficiencies and increases in chromosome abnormalities and malignancies.

As such, telomeres and telomere driven senescence may lead to exhaustion of cellular reserve and repair capacity represents a causative factor in the aging process and age related pathologies. Additionally, the manifestation of dysfunctional and shortened telomere phenotypes in lower-turnover tissues, such as the lung, demonstrates that telomere influence extends beyond high turnover tissues and further underscores the importance of considering tissue specific contexts within telomere and aging biology.

The strong association between telomeres, cellular proliferative potential and age related pathologies has resulted in the investigation into telomere length as a biomarker, particularly in relation to biological versus chronological age, disease risk or mortality and environmental and lifestyle influences.

In humans, core relationships between age, gender and race have been shown with age-matched males and Caucasians appearing to have shorter telomeres on average compared to females and other races respectively. Beyond this base, correlations between telomere length and malignancy, obesity, glucose metabolism, cardiovascular disease, smoking and stress have been demonstrated. Studies have also reported links between telomere length and variables such as health status, exercise levels, psychological adversity, meditation, cognitive capacity, and socioeconomic and social standing. In relation to overall mortality, there is clear inconsistency between studies, with some reporting relationships between short telomere lengths and risk of death or remaining lifespan, while others reporting a failure to find comparable significance or results. For example, some twin studies have found that the twin with shorter mean telomere length experienced an increased risk of mortality within the timeframe of the study.

Despite notable success, there are also obvious limitations to the conventional use of telomere length as a biomarker. Due to conventional methodological limitations and choices, most current studies use only single telomere values, usually mean telomere length, to represent individual participants. As each cell has two telomeres per chromosome arm, this simplification has the effect of removing considerable information and power from comparisons and studies. Additionally, despite evidence telomere length and biology varies between tissue and cell types, most biomarker studies rely solely on peripheral blood samples to determine telomere length. Therefore, tissue differences, as well as their relationships to measured outcomes, may be further confounding analysis and obfuscating some of the results presented by studies to date.

Additionally, abnormal telomere biology has been extensively linked to oncogenesis. Specifically, the evasion of senescence and telomere proliferation control by telomerase or ALT activation is a critical step in malignancy and cellular immortalization. Accordingly, telomerase activity can be detected in approximately 90% of all malignant samples and high levels of expression indicate a poor prognosis. The remaining approximately 10% of samples rely on the ALT pathway for continued telomere extension and proliferation. There is evidence shortened telomeres predispose cells for oncogenic transformation and associations between prognosis and telomere length, telomerase levels and ALT phenotype have also been demonstrated. Functionally, continued cellular proliferation with unstable or critically short telomeres can drive genomic instability and potential oncogene amplification. Thus, determining the detailed telomere lengths can be an asset for examining oncogenesis.

In view of the power of knowing telomere length, as demonstrated in the foregoing, techniques, and variations thereof, have been developed for telomere length measurement. The variety and relevance of such techniques can be traced in part to specific telomere features, such as repetitive elements, multiple telomeres per nucleus and potentially meaningful chromosomal, cellular and individual variation. These characteristics present inherent challenges and can lead to considerable compromises during measurement depending on the strategies and techniques used. As a result, measurement techniques are an important consideration when investigating, diagnosing and treating telomere biology.

In general, telomere length measurement typically includes a probe specific to the telomere repeat sequence and can include additional probe(s) or stain(s) to identify specific telomeres or the chromosome on which the telomere is located. Telomere length measurement also typically includes an illumination and capture device suitable to capture the information from the probes. In some cases, a system can be used to convert the raw measurements of the probes into quantitative measurements for analysis.

One such technique for telomere length measurement is the terminal restriction fragment (TRF) assay, which uses southern blotting and a telomere specific probe to determine a general telomere length distribution for each sample. Prior to comparison between samples, these distributions are further simplified to single values, such as mean telomere length. As the telomeres from all chromosomes and cells are combined into a single distribution, TRF can only give limited insight into more detailed chromosome and cell specific telomere dynamics. Additionally, comparisons between studies and species are complicated by variations in subtelomere structure and TRF methodology.

Another technique for measurement of mean telomere length can be obtained with a quantitative polymerase chain reaction (qPCR) based approach. Using specially designed primers and Real Time PCR, it is possible to compare the generation of telomere (T) and single copy reference gene (S) amplification products. Changes in telomere length alter the amount of telomere template available during amplification cycles resulting in a corresponding change in the T/S ratio. The qPCR approach only offers mean telomere length values as an output.

Single telomere length analysis (STELA) is another PCR-based approach. In this case, chromosome specific subtelomere primers and specialized telomere linkers provide measurements for individual telomeres, based on primer specificity, within the available template DNA. While this approach gives chromosome specific data, it is limited to the few telomeres where suitable subtelomeric primers are available and excludes any information on the relationships between the lengths of the different telomeres of the same cell. This approach also combines information from multiple cells. STELA telomere length distributions tend to be simplified to individual component values or other partial metrics before comparisons between samples.

Another technique for telomere measurement can be using fluorescence in situ hybridization (FISH) and flow cytometry. This approach, known as flow FISH, uses a fluorescent probe that specifically recognizes and binds to telomeric DNA sequences allowing for their quantification using flow cytometry. Flow FISH involves the measurement of telomere length across large numbers of cells, but also averages telomere length to a single value for each cell.

Flow cytometry uses a flow cytometer; a device that ‘flows’ a sample past one or more light sources and detectors that measure the interaction between the sample and the one or more lights. Used with FISH, Flow FISH can be used to measure telomeres directly.

A related approach, chromosome flow FISH, partially overcomes the limitations of Flow FISH by identifying specific chromosomes and performing measurements on individual chromosomes. However, not all chromosomes can be resolved with conventional protocols for this approach and any data on relationships between the telomere lengths of different chromosomes within individual cells is not provided. In addition, flow FISH has considerable technical challenges and increased costs compared to other techniques.

Another technique for telomere measurement is using quantitative FISH (qFISH). qFISH involves FISH of a telomere specific probe to fixed cells either in interphase (interphase qFISH) or metaphase qFISH (mqFISH). In the qFISH approach, greater fluorescence can represent more of a specific DNA sequence being present. It is noted that qFISH typically refers to the combination of FISH and microscopy-based image capture, followed by quantification of the images. However, in some cases, qFISH could also refer to quantification of FISH signals by other equipment and/or means; for example, flow FISH may be considered a form of qFISH because it involves the quantification of FISH signals by a flow cytometer.

Interphase qFISH shares the same limitations as Flow FISH, along with lower throughput and a general limitation to use in fixed samples.

Given high quality preparations, mqFISH allows for the simultaneous visual identification of chromosomes and quantification of their associated telomeres within single metaphases. Thus, mqFISH is able to give information on the relationships between chromosome specific telomere lengths within a single cell. As metaphase preparations are central to mqFISH, proliferating cells, for example stimulated lymphocytes or fibroblasts, are typically required.

Using the mqFISH technique, visual representations can be captured and converted into quantitative telomere measurements. This technique can use a fluorescent light source to illuminate a sample; for example using an arc lamp, mercury-vapor lamp, LED, or laser. In some cases, appropriate optical filters are used to help suitably illuminate the sample and isolate the fluorescent signal. The mqFISH technique can also use a microscope. The microscope can be used with a camera, and in some cases camera software, that captures an image of the fluorescent signal emitted by the probe and/or sample. The mqFISH technique can also use image analysis, for example executed on a processor, which converts the brightness of the pixels for the probe spots captured in the image into quantitative measurements. Typically, these measurements take into account or otherwise correct for the brightness of ‘background’ pixels of the image. In some cases, the mqFISH technique can also use executed software that assists in the preparation of a karyotype; for example via an organized table of the chromosomes in the captured image. In some cases, the fluorescent light source and microscope could be grouped together as a fluorescent capable microscope imaging station. Such a station typically includes the microscope with an appropriate light source, optics and camera, and a connected computing module with output device for capturing, processing, storing, and displaying the images. The image analysis and karyotype software can each be standalone applications or can be incorporated into the same software application that also captures the image.

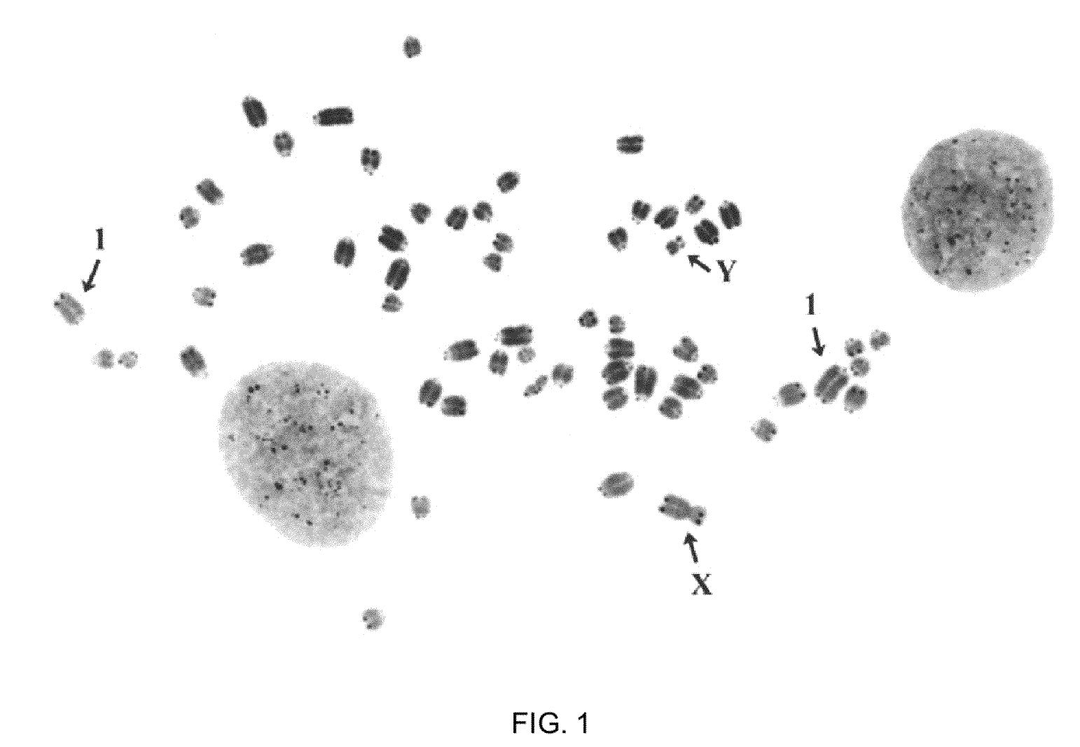

As an example of chromosome identification and telomere visualization using qFISH, FIG. 1 shows a bovine quantitative fluorescence in situ hybridization image displaying a complete metaphase of 60 chromosomes as well as two interphase nuclei (the colours of the image have been gray-scaled and inverted for reproducibility). Telomeres are visualized using a (TTAGGG)n specific fluorescent probe, with larger and brighter spots representing longer telomeres; DNA is visualized using a DAPI counterstain. The largest autosome, chromosome 1, and the metacentric sex chromosomes, X and Y are marked. For bovine qFISH, the shared acrocentric classification and similar sizes of the bovine autosomes means there is some difficulty in differentiating between them beyond identification of chromosome 1 when using DAPI counterstaining alone.

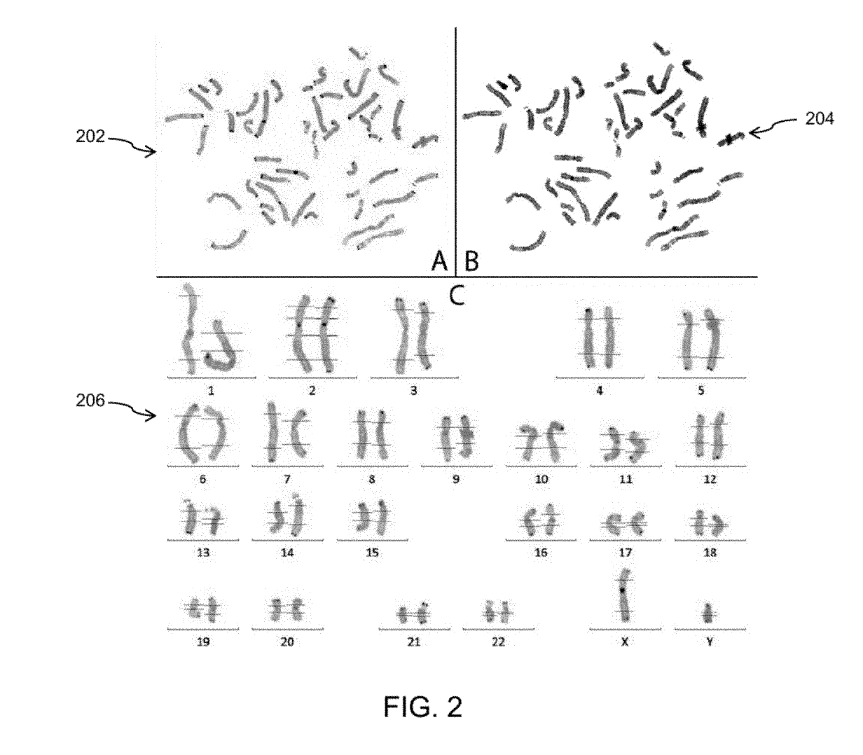

For the mqFISH technique, high quality metaphase preparations are required and a specialized peptide nucleic acid (PNA) probe, or a similar equivalent, must be used in place of conventional DNA probes to provide the specificity, robustness and reliability necessary for quantitative measurements. Careful imaging, followed by computational analysis, can be used to determine the size and brightness above background for each telomere signal. Such size and brightness can be used to quantify telomere length. Particularly, the more repeats a telomere has, the more probe it can bind and the brighter and larger the fluorescent signal; see, for example, FIG. 2. Biological and technical variability necessitates the measurement of multiple metaphases for each sample. In some cases, the inclusion of a centromere specific PNA reference probe reduces assay variation and facilitates the comparison of telomere length between different cells and samples. In some cases, calibration to a telomere signal of known length may be used to convert mqFISH values into base pair units.

FIG. 2 shows an exemplary sample of XY human mqFISH metaphase 202 with FITC telomere signals (the colours of the image have been gray-scaled and inverted for reproducibility). This example has a chromosome 2 centromere reference and added X centromere signals from supplementary PNA probes and has a DAPI DNA stain overlaid. Inverted DAPI banding 204 is used in this example to identify individual chromosomes and prepare a karyotype 206 to allow for the measurement and collection of chromosome specific telomere lengths. Overlapping chromosomes (5 and 9) have been digitally separated in this metaphase image to facilitate karyotype placement. The approximately horizontal lines in 206 delineate each FISH signal. P-arm telomere signals are located above the upper horizontal line, chromosome reference signals are located between the two horizontal lines and q-arm telomere signals are located below the lower horizontal line. Quantification of individual signals, using mqFISH, is accomplished by image processing and analysis that takes into account intensity above background at each pixel, and assigns a relative total value based on that intensity.

The same aspects that can complicate telomere length measurement can also contribute significant complexity to its analysis. In most assays, the information contained in chromosome arm, pair and cell specific telomere length relationships is simplified. The resulting analysis may become more straightforward, and may not require reconciling or combining the multiple related values for each sample; however, in these cases, the power and ability to gain further insight into telomere biology is also lost. For example, the TRF assay analysis may only require comparison of a single telomere length distribution, or even just a single value, between samples, but such analysis cannot give any insight into chromosome specific telomere length dynamics. As well, such analysis can only minimally reveal the heterogeneity of telomere length within each sample. In addition, the loss of detail accompanying simplifications can lead to a requirement for significantly larger sample sizes during certain types of comparisons.

Using mqFISH, chromosome specific telomere length data, as well as details on the relationships between chromosome pairs and arms, can be determined. Some approaches using mqFISH consider only the information mqFISH provides in isolation; for example, telomere 9p in sample A versus 9p in sample B. As well, some approaches only aggregate the data before comparisons, which leads to the exclusion of information from the comparison despite it being present within the dataset. As an example, if the two telomere 9p values for a single metaphase are simply averaged before comparison to another sample or metaphase, the approach excludes information that one value represented the paternal chromosome and the other value the maternal chromosome in each metaphase.

As described in the forgoing, the length of telomeres is dynamic and has demonstrated, or putative, associations with a considerable number of cellular, physiological, genetic, epigenetic and disease characteristics; most notably to aging and future cellular proliferative capacity. With 2 telomeres per chromosome per cell in which to extract information, telomere length and patterning can be a powerful biomarker. In conventional approaches, features such as homologous chromosomes, the number of individual telomeres in a single cell and the normal variability intrinsically present in telomere lengths adds complexity that is overlooked or not suitably addressed.

Typically, conventional telomere analysis simplifies the telomere analysis. Under such conventional approaches, the use of telomere length as a biomarker suffers from ill-suited analysis methodologies that inefficiently utilize available data and can obfuscate results and comparisons. When considering and comparing potential complex relationships between samples, conventional techniques typically rely on singular or isolated points of comparison between measurements. As an example, consider a data set consisting of ABCDEFG measurements for two samples, S1 and S2. For a comparison of S1 to S2 using conventional techniques, a comparison can be made of S1 A measurements to S2 A measurements, and S1 B measurements to S2 B measurements, and so on. However, such individual comparisons are not suitable to be aggregated or combined to give an overall comparison of S1 to S2. As a further example of conventional approaches, consider comparing S1 and S2 by combining all the lengths ABCDEFG of each sample into a single combined A-G measurement and then comparing S1 A-G to S2 A-G. As in these examples, conventional approaches generally fail to facilitate a complete and detailed comparison between S1 and S2, especially if there are potential relationships between the various A-G measurements; as is often the case in biological data.

Additionally, conventional approaches considerably compromise comparison and biomarker utility by either discarding or failing to aggregate potentially informative information. As an example, TRF-Southern Blotting combines all telomere lengths on the lab bench and only a single distribution is available for comparison between samples. In another example, while mqFISH allows for the collection of comprehensive data on telomere length at the level of individual telomeres, conventional analysis has generally focused on the longest and shortest telomeres, and has failed to aggregate results to the point of comprehensive comparisons between samples. This lack of a robust approach for efficiently capturing, integrating, comparing, and generally analysing telomere length data has greatly compromised the full potential of telomere measurement, for example with mqFISH, and its greater use.

In some cases, more advanced approaches, such as regression analysis, can be employed. These approaches are typically used in an attempt to make more detailed comparisons for certain situations. However, such approaches typically have significant limitations and trade-offs. In the above example with two samples S1 and S2, regression analysis would allow a comparison between S1 and S2 that considered measurements ABCDEFG of each sample individually before combining them to an overall comparison; however, such an approach would do so in a simplifying manner and would not capture potential relationships between the A-G measurements. Such conventional approaches can also be inflexible in terms of making meaningful adaptations to the analysis for different samples, conditions or conclusions. In addition, such conventional approaches can make it difficult or impossible to draw parallels between the individual relationships or subcomponents within the analysis and the data or biological system itself. As such, such conventional approaches are limited in the scope, utility and understanding that can be achieved.

The system and method, as described herein, utilizing the benefits of computational power, has the advantage of being able to capture, aggregate and utilize additional patterning information, such that it is able to compare the chromosome specific telomere lengths of reprogrammed cells to the donor cells from which they are derived. In this way, the Applicant recognized the substantial advantages of using computational power to allow for more powerful, objective and quantitative analysis for comparisons for telomere length analysis. The system and method, as described herein, allows for individual comparisons between significant quantities of measurement data to be made and then aggregated upwards in an objective and quantitative manner to make meaningful higher level, or overall comparisons and analysis between samples.

With reference to the above example of two samples, S1 and S2, the system and method, as described herein, powerfully allows S1 A to be compared directly to S2 A, and S1 B to be compared directly to S2 B, and so on. It is intended that such an approach also provides a means for all of the individual comparisons to be combined in order to provide a full comparison of S1 to S2. Applicant recognized the substantial advantage of this approach of being able to capture both the details of individual comparisons and broader comparisons between S1 and S2. Such broader comparisons can occur without having to simplify away the individual details or being limited to looking at each measurement in isolation.

Additionally, the flexibility of the approach of the system and method, described herein, allows additional components to be generated from the original measurements and included in the comparison analysis. These additional components allow for information on potential complex relationships between the different measurements to be captured and included in analysis and comparisons. As an example, component AB may consist of the normalized ratio of measurement A to measurement B. This component would capture a potential relationship in the values between measurement A and measurement B and facilitate the comparison of that relationship between S1 and S2 in a way that simply looking at S1 A versus S2 A and S1 B versus S2 B alone may have missed. Such an approach is flexible, and as many components as are appropriate to capture the potential relationships can be propagated and integrated into the analysis. Therefore, such an approach allows for both multiple measurements to be compared and for more complex relationship or patterns to be included in the comparisons, via the use of components.

The Applicant recognized the substantial advantages of an approach that allows for the information present in telomere measurement techniques, such as mqFISH, to be analyzed and compared by a system in a holistic and integrated manner between samples. The approach, as described herein, has the ability to better take advantage of the information present in measurement data, for example mqFISH data, during inter-sample comparisons. In this way, a significant obstacle to the greater use of mqFISH can be overcome with computational approaches and a greater realization of its experimental and comparative potential can be harnessed.

Telomere length at each chromosome arm can represent a combination of organism specific characteristics, cell type epigenetic influences, and accompanying alterations accrued during the biological and stochastic history of the cell. Due to the function and physiology of telomeres, the maintenance or alteration of chromosome specific telomere length characteristics could impact long-term genetic and proliferative stability following epigenetic reprogramming procedures. Accordingly, the Applicant recognized the significant advantages of observing and comparing telomere length patterns at the chromosome specific level in a reprogramming context. In this way, through use of the system as described herein, new insights into telomere biology and the epigenetic reprogramming process, as well as other aspects of cellular biology and phenotype through their overlapping relationships with telomeres and telomere length, can be understood.

In an exemplary application of the system and method, as described herein, they can be used for making an observable determination of similarities in chromosome specific telomere length patterning between tissue-matched donor and reprogrammed cells. The Applicant recognized the substantial advantages of developing an approach for making pattern based telomere length comparisons that incorporate multiple telomere characteristics, which can be used to test the above similarities. Such approach can significantly increase the utility of telomeres as an experimental tool and biomarker.

The Applicant further recognized the advantages, using the system and method described herein, of capturing potential differences in telomere lengths between chromosome pairs, or maternal and paternal chromosomes, and determining distributions of the lengths in the comparisons. As an example, this information can be used during sample comparisons. Further, using the system and method, as described herein, the Applicant recognized the advantages of facilitating overall comparison between samples and sample groups without loss of detail; and to allow each individual variable to be quantitative and made in a manner that allowed for objective aggregation to higher-order comparisons.

Additionally, the Xi chromosome displays exaggerated telomere and epigenetic characteristics and X-inactivation status is known to play an important role in reprogramming outcomes. In this way, the system and method, as described herein, includes the substantial advantage of allowing for more detailed analysis and observation of the Xi chromosome and its telomeres.

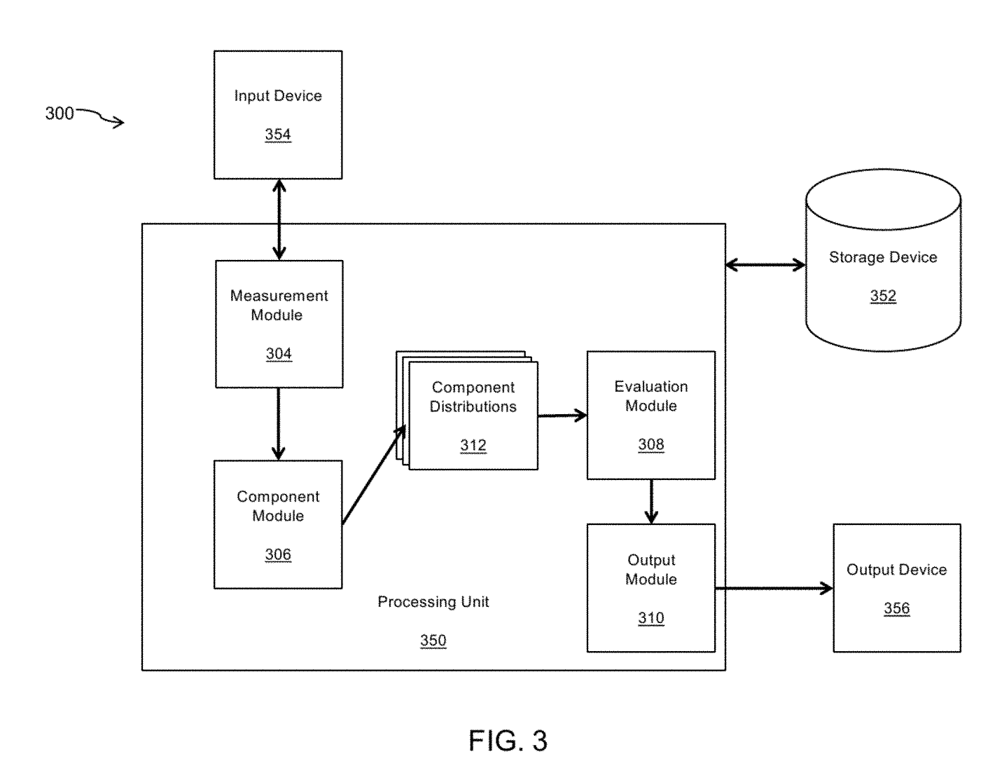

Turning to FIG. 3, a block diagram for a system for patterning analysis of a telomere length dataset 300 is shown. The system for patterning analysis of a telomere length dataset 300 includes a processing unit 350, a storage device 352, an input device 354, and an output device 356. The processing unit 350 includes various interconnected elements and modules, including a measurement module 304, a component module 306, an evaluation module 308, and an output module 310. The processing unit 350 may be communicatively linked to the storage device 352 which may be loaded with data, for example, measurement data, component distribution data, or comparison data. In further embodiments, the above modules may be executed on two or more processors, may be executed on the input device 354 or output device 356, or may be combined in various combinations.

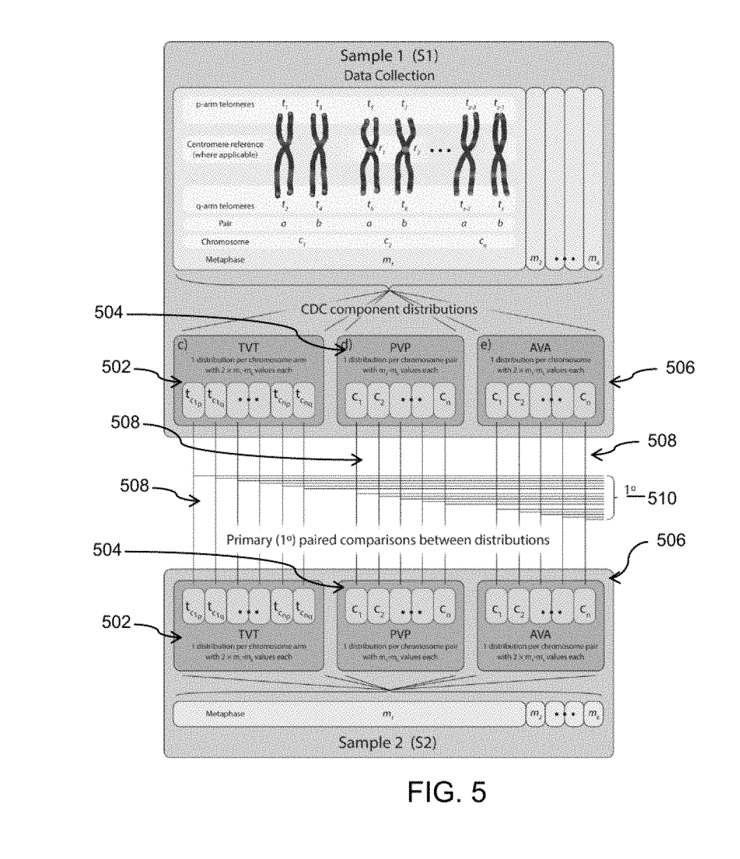

To capture the telomere information, the system 300 extracts and decomposes the relationships within the telomere information into, for example, three chromosome specific components. The three chromosome specific components being the telomere, pair, and arm. In further cases, the system 300 can extract and decompose into less than three, or more than three, chromosome specific components. The system 300 can then objectively evaluate the components in a manner that allows for the logical aggregation of the results and quantitative comparisons between samples, as described herein; see, for example, the diagram of FIG. 5.

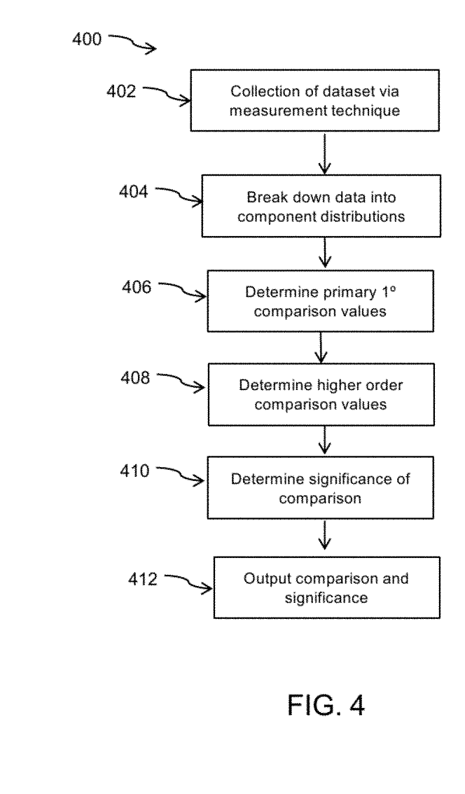

Turning to FIG. 4, a method for patterning analysis of a telomere length dataset 400 is shown. At block 402, the measurement module 304 captures a dataset using a measurement technique, in this case mqFISH, via the input device 354. The input device 354 includes suitable equipment for taking the measurement, such as an imaging device, and may include suitable device for computational analysis, such as a processor, to work with the measurement module 304, such as to determine the size and brightness above background for each telomere signal. In further cases, the computational analysis can be executed solely by the measurement module 304, or another suitable module on the processing unit 350, or solely by the processor located on the input device 354. In further cases, the measurement module 304 could be adapted to capture data from other measurement techniques, such as chromosome flow-FISH.

In most implementations of the mqFISH technique, the measurement module 304 captures the data such that each metaphase is captured within a single frame and with even illumination across the frame. In some cases, the measurement module 304 can also optimize the illumination and exposure timing for each individual metaphase to ensure telomere and reference signals are not under or over exposed. In some cases, the use of digital gain or any post-processing is avoided as it may be non-linear. A minimum number of metaphases should be used for increased accuracy and to provide detail on the variation in telomere lengths between individual cells within the sample; preferably, a minimum of 10 metaphases per sample is used. Larger numbers of metaphases, and thus larger sample sizes, are preferable when possible.

In some cases, the measurement module 304 can include normalization with a centromere reference probe. In other cases, where no reference probe is available, a variation using fractional values, as described below, can be employed.

In some cases, where a full karyotype cannot be reliably identified using DAPI counterstaining, such as the case with lower quality samples or with some non-human species, CDC analysis can be carried out on the subset of identifiable chromosomes alone.

The measurement module 304 preferably measures signals from captured images taking into account background fluorescence, the size of the signal, and the intensity of each pixel.

While biological differences may exist, telomere variation between sister chromatids of metaphase chromosomes with the mqFISH measurement technique may be more technical than biological in nature and therefore in some cases are not included. In some cases, if measured separately by the measurement module 304, the values for distinct chromatid signals can be merged. The measurement module 304 can record the sample, metaphase chromosome, arm and homologue identifiers associated with each raw telomere value. In most cases, homologue identifiers do not consistently identify the same homologous chromosome between metaphases, but serve to distinguish between the two homologues within a single metaphase during the analysis.

In cases where the measurement module 304 uses a centromere reference probe, the average of the two centromere measurements in each metaphase may be used to normalize the raw telomere measurements of that metaphase. As an example, using the following normalization formula:

$${Normalization}_{Completed per metaphase} normalized {t}_{x} value ( {}_{n}^{}{t}{}_{x}^{} ) = log \frac{{t}_{x}}{( {r}_{1} + {r}_{2} ) + 2}$$

In cases where the measurement module 304 does not use a centromere reference probe, the raw measurements for each telomere may be normalized as necessary by converting them into a proportion of the sum of all telomeres considered in each given metaphase and component distribution (as described below).

In some cases, telomere measurements can be further normalized to the mean telomere value per metaphase to put greater focus on differences in telomere length between the chromosomes versus overall telomere length.

At block 404, the component module 306 determines complex patterning relationships between telomeres of the dataset captured by the measurement module 304 and disposes them into generated component distributions 312. In the present embodiment, the relationships are broken down into the following three component distributions: 1) telomere versus telomere (TVT) having 2 values per chromosome per metaphase, as shown in FIG. 5 at element 502; 2) pair versus pair (PVP) having 1 value per chromosome pair per metaphase, as shown in FIG. 5 at element 504; and 3) arm versus arm (AVA) having 2 values per chromosome pair per metaphase, as shown in FIG. 5 at element 506. These component distributions are intended to capture information relating to the telomere value itself (TVT), the relationship and differences in telomere values between each chromosome pair (PVP), and each chromosome's p and q arms (AVA), respectively. Such information can then be analyzed for differences between samples in a pairwise manner.

In cases with a centromere reference, the TVT, PVP and AVA component values can be determined (respectively) as follows:

| | |
|---|---|
| TVT | TVT_{c}_{₁ₚ}= ₙt₁ & ₙt₃ |
| 2 values per telomere | |
| PVP | PVP_{c1} = \|log[(ₙt₁ + ₙt₂) + (ₙt₁ + ₙt₂)]\| |
| 1 value per pair | |
| AVA | AVA_{c1} = \|log(t₁ ÷ t₂)\| & \|log(t₃ ÷ t₄)\| |
| 2 values per pair | |

In cases without a centromere reference, the TVT, PVP and AVA component values can be determined (respectively) as follows:

$$\begin{matrix}{TVT}_{2 values per telomere} & {TVT}_{{c}_{1 p}} = log ( {t}_{1} + Σ {t}_{1 - z} ) \& log ( {t}_{3} + Σ {t}_{1 - z} ) \\ {PVP}_{1 value per pair} & {PVP}_{c 1} = \vert log \frac{( {t}_{1} + {t}_{2} )}{( {t}_{3} + {t}_{4} )} \vert \\ {AVA}_{2 values per pair} & {AVA}_{c 1} = \vert log ( {t}_{1} + {t}_{2} ) \vert \& \vert log ( {t}_{3} + {t}_{4} ) \vert\end{matrix}$$

In the above examples, log transformations can simplify interpretations of the relationships between values within the component distributions.

Once the component module 306 populates the component distributions with the appropriate values, the evaluation module 308 can perform comparison and aggregation analyses on the component distribution information. At block 406, the evaluation module 308 determines primary 1° values. In some embodiments, at block 408, the evaluation module 308 can then aggregate the primary 1° values and determine secondary 2° or further higher order comparisons.

As an example, primary 1° comparisons are diagrammatically shown as element 508 in FIG. 5. The evaluation module 308 can use non-parametric Anderson-Darling (AD) assessments, or other suitable statistical assessments, to make pairwise evaluations between each individual component value in the component distribution and a sample pair. The AD assessment allows the determination of a quantitative value representing the level of similarity and dissimilarity between the two distributions. The AD assessment is sensitive to differences in both the range and overall shape of the component distributions. With the AD assessment, every individual component value in the component distribution, as well as the variation of the collective entry values in the component distribution, informs the component module's 306 comparisons. The result of each pairwise AD assessment is an output statistic, or primary (1°) comparison value, with larger values representing greater dissimilarity. Once all pairwise AD assessments have been completed, the primary 1° values, and their corresponding critical values, can be aggregated as appropriate by the evaluation module 308 for secondary (2°) or further higher order assessment. Higher order assessment by the evaluation module 308 can determine higher order values with higher order comparison analysis that can determine significant differences between samples. Such an aggregation of primary 1° values 508 for a secondary 2° comparison is diagrammatically shown as element 510 in FIG. 5. Advantageously, the pairwise assessment allows the primary 1° values to be further aggregated while preserving their quantitative nature, facilitating higher order assessments and analysis, as described herein.

As an example, a secondary 2° assessment can include, with larger values representing a greater degree of dissimilarity, one or more of the following:

$$Full comparison of telomere similarity between S 1 \& S 2 = Σ 1 {°}_{1 \& 2}$$

$$Chromosome with the most telomere dissimilarity between S 1 \& S 2 = max Σ ( 1 {°}_{S 1 \& S {2}_{{TVT}_{{c}_{xp}}}} + 1 {°}_{S 1 \& S {2}_{{TVT}_{{c}_{xq}}}} + 1 {°}_{S 1 \& S {2}_{{PVP}_{{c}_{x}}}} + 1 {°}_{S 1 \& S {2}_{{AVA}_{{c}_{x}}}} ) \\ Telomere with the most dissimilarity between S 1 \& S 2 = max Σ 1 {°}_{S 1 \& S {2}_{{TVT}_{{c}_{{x}_{y}}}}}$$

In another embodiment, the evaluation module 308, at block 406, can use a Kolmogorov-Smirnov (KS) assessment instead of the AD assessment. Preferably, only one of the AD or KS assessments should be used for all component comparisons in a given evaluation, as specific values obtained from one of the AD or KS assessments cannot be aggregated or meaningfully compared with the other.

The AD assessment is generally more sensitive to differences towards the edges of the component distribution, while the KS assessment is more sensitive to the central portion of the distribution. In cases with unequal metaphase numbers, the evaluation module 308 can weigh the primary 1° values as appropriate to maintain equivalence during aggregation. In further cases, obtaining fairer comparisons in studies containing both female and male samples may involve the evaluation module 308 excluding specific primary 1° values related to the sex chromosomes and weighting the remainder.

In an example, where the measurement module 304 uses a centromere reference, 10 human female samples of 25 metaphases each are captured. In this case, there are 94 raw measurements, consisting of 92 telomere and 2 centromere measurements, for each metaphase. There would thus be 23,500 total measurements. Each sample would include 46 TVT, 23 PVP and 23 AVA component distributions populated with 50, 25 and 50 individual component values respectively. Full paired comparison of all 10 samples by the measurement module 304 gives 1,104 primary 1° values.

As equivalent AD tests have been performed, the primary 1° values can be objectively and logically aggregated by the evaluation module 308 to make meaningful and interpretable comparisons and conclusions between the samples. For example, the evaluation module 308 can determine a quantitative ranking and comparison of telomere similarity. Such ranking and comparison can be obtained by setting one sample as a sample pair reference, obtaining the sum of the corresponding 92 primary 1° values for the other 9 samples, and then ordering the sums. If the 9 other samples represent 3 groups of 3 samples each, then aggregating the 276 primary 1° values for each group by the evaluation module 308 can provide a direct and quantitative comparison between the sample groups. The sample pair reference can be chosen at random or chosen by predetermined selection criteria.

The evaluation module 308 can also determine chromosome pairs or telomeres with the most and least similarity across all or a subset of samples by selecting and aggregating the pertinent primary 1° values.

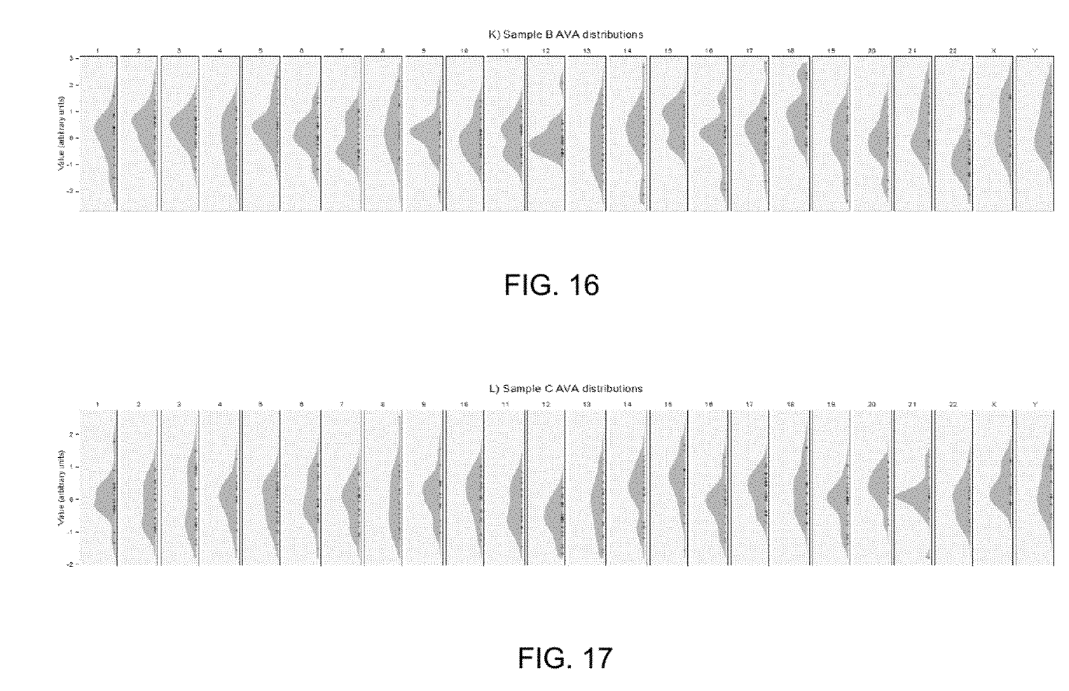

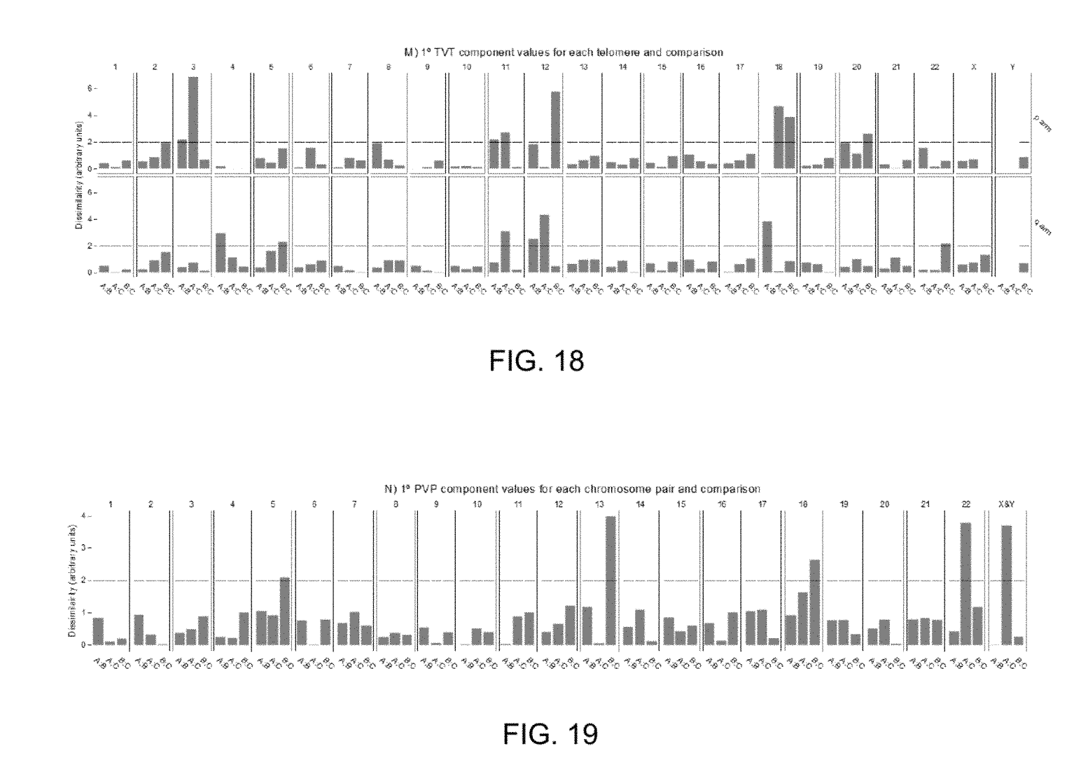

At block 410, in some embodiments, the weighting module can assign a measure of significance to the comparison values determined by the evaluation module 308. The measure of significance can be obtained according to a suitable approach based on the data determined by the evaluation module 308 and what is suitable for a given application. Primary 1° value distributions, and aggregations for samples and groups, can each be assigned a significance measure of their own. In some cases, primary 1° values can be filtered on the basis of individual significance during aggregation. In further cases, as exemplified in FIGS. 18 to 20 and 24 to 27, corresponding values for critical significance can be aggregated along with the primary 1° values and used as guide for a cumulative significance measure. Preferably, sets of reference samples or control experiments can be included, where possible, to provide context in the form of natural distributions of component and primary 1° values.

At block 412, the comparisons and statistics determined by the evaluation module 308 are outputted to an output device 356 via an output module 310. The output device can be any suitable device, for example, a computer monitor or tablet display.

Applicant recognized the significant advantages over conventional telomere length analysis techniques. As an example, the use of the TVT, PVP and AVA components, and the AD or KS assessment of such components, offers a technological solution that provides for more powerful comparisons and data extrapolation by capturing data from chromosome specific relationships and distribution variation. Further, logical aggregation of pairwise sub-comparisons has the substantial advantage of facilitating meaningful, easy to interpret, and quantifiable analysis between samples and sample groups. Furthermore, the system and method described herein is adaptable to telomere data sources beyond the mqFISH measurement technique.

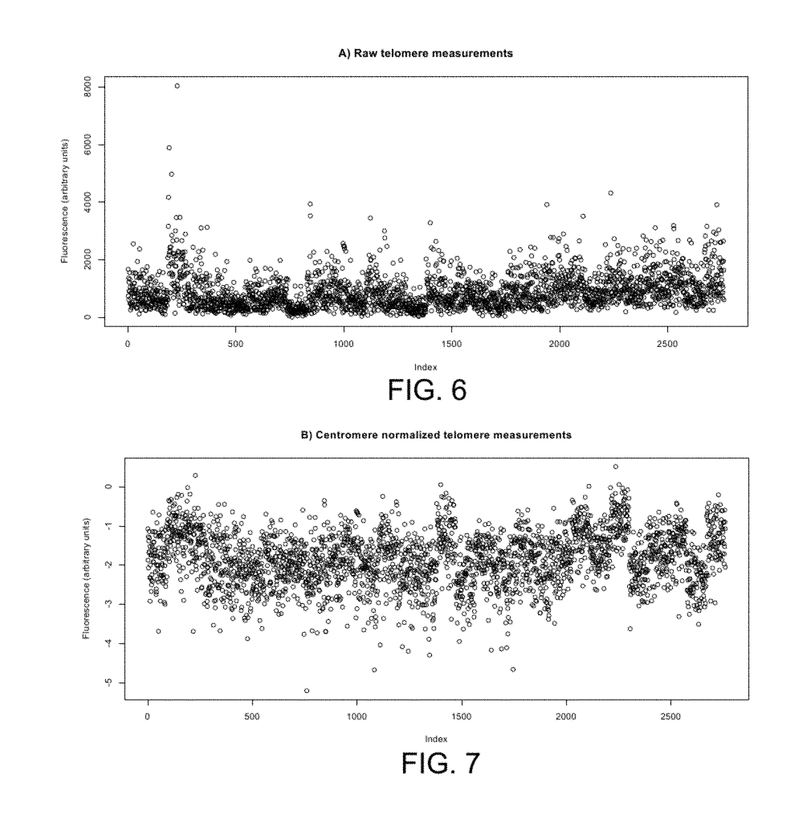

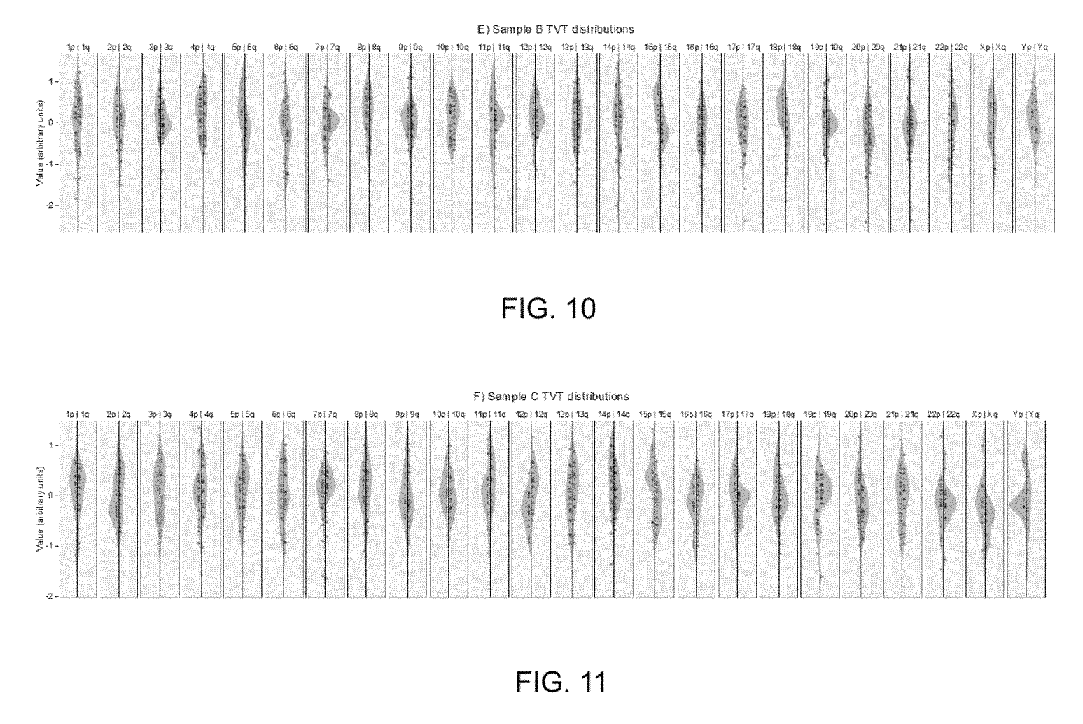

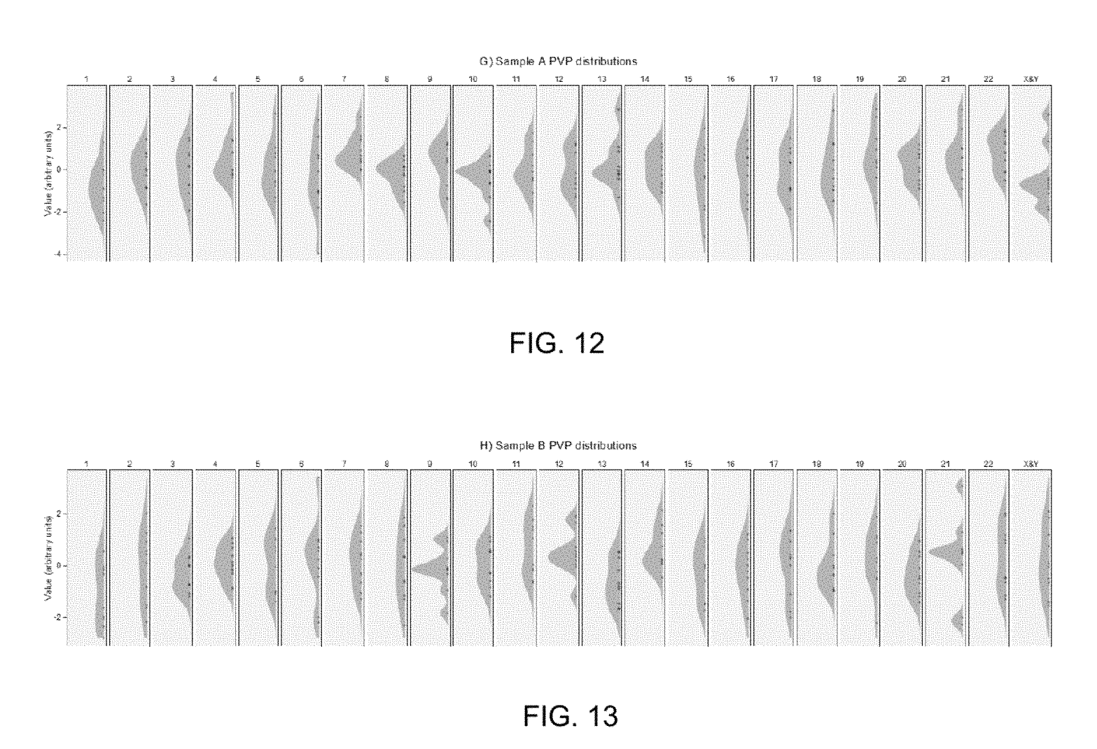

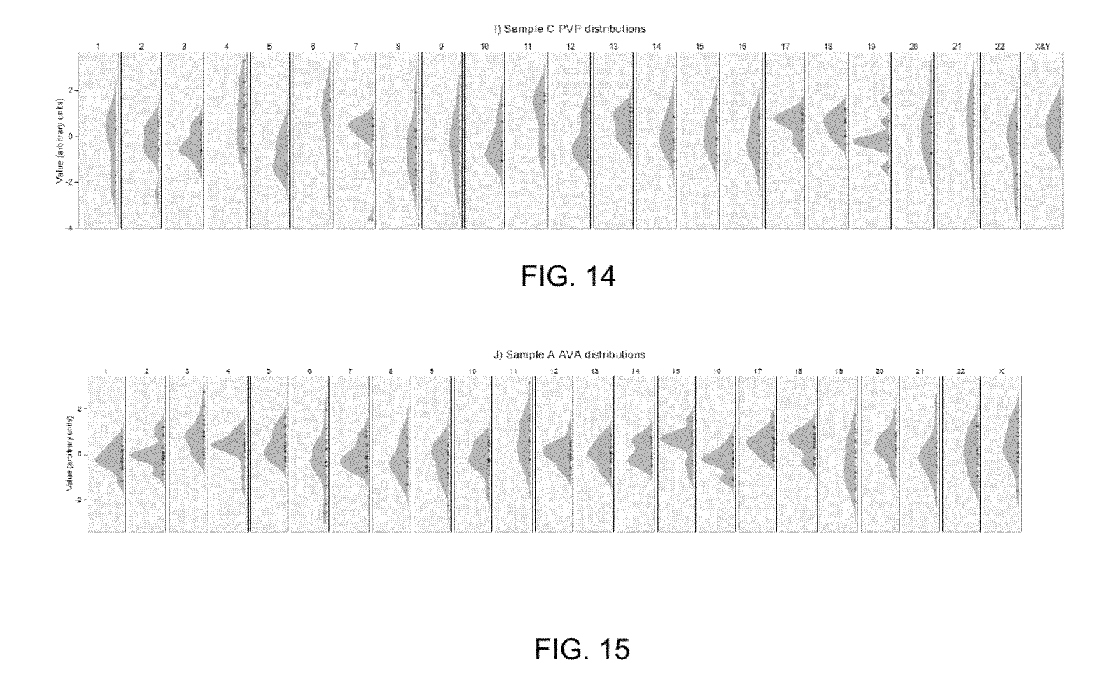

FIGS. 6 to 24 show an example of the method 400, evaluating raw data into aggregated comparisons. This example includes 3 human samples, 1 female (Sample A) and 2 male (Sample B and Sample C).

In FIG. 6, raw measurements for all 92 telomeres and 2 reference centromere signals are compiled by the measurement module 304 from 10 high quality mqFISH metaphases per sample. In FIG. 7, to allow further comparison between metaphases, the raw telomere values (n=920 per sample) are normalized on a per metaphase basis using the mean of the 2 centromere reference signals. When centromere reference probes are unavailable, an alternative approach where each telomere is considered a fraction of the summed telomere values under comparison can be used instead.

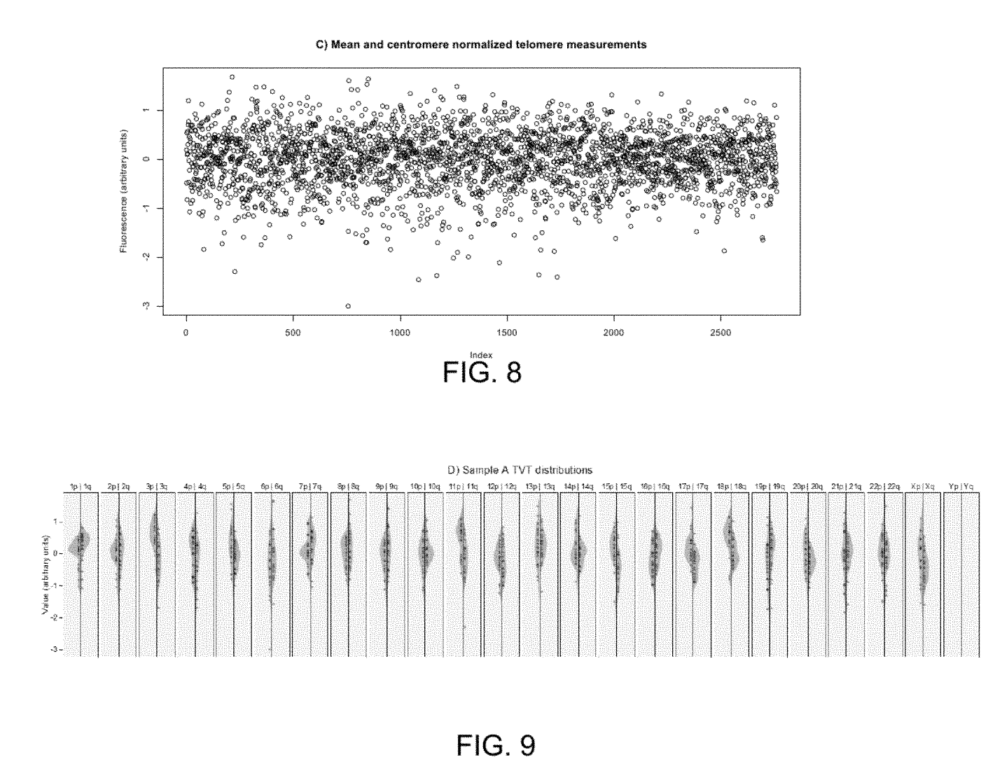

In some cases, as illustrated in FIG. 8, to put greater focus on differences in telomere length between the chromosomes rather than the overall telomere length differences between metaphases and samples, a further normalization to the mean telomere value, or similar value, can be applied on a per metaphase basis.

The relevant normalized values are used, by the component module 306, to populate, for each sample, the TVT component distributions, PVP component distributions and the AVA component distributions. TVT component distributions are shown in FIGS. 9, 10 and 11, with the 920 values per sample divided into 48 or 46 distributions for male and female samples respectively. PVP component distributions are shown in FIGS. 12, 13, and 14, with the 230 values per sample divided into 23 distributions. The AVA component distributions are shown in FIGS. 15, 16, and 17, with the 460 values per sample divided into 24 or 23 distributions for male and female samples respectively.

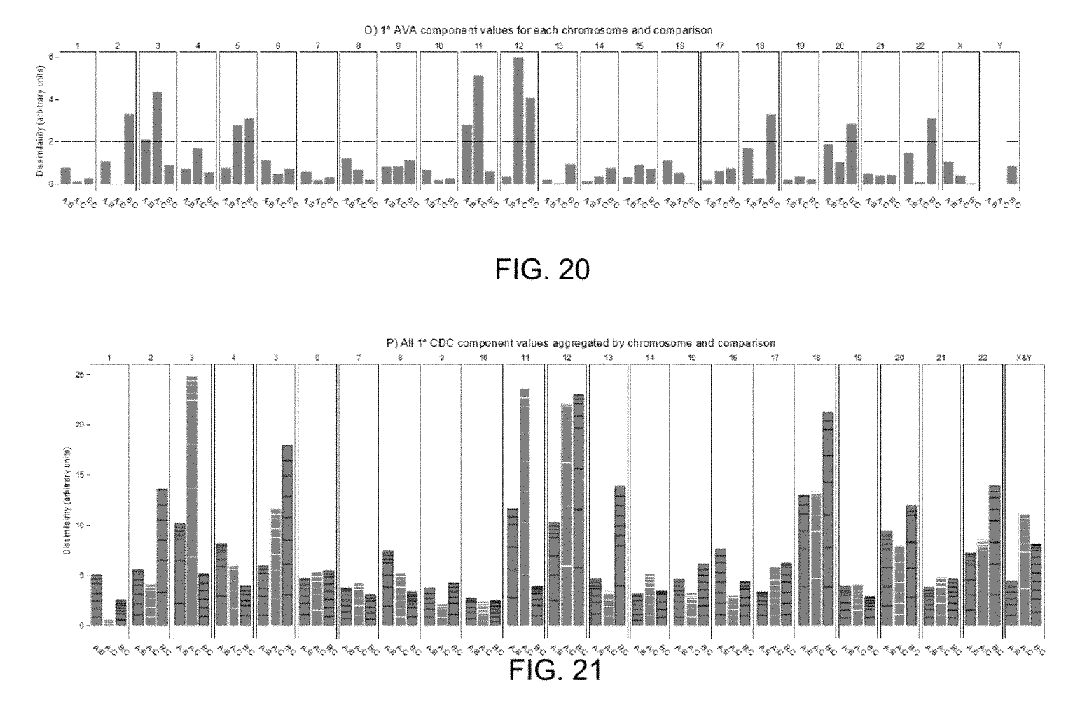

The evaluation module 308 can then provide a comparison analysis of the populated component distributions in a pairwise manner using either Kolmogorov-Smirnov (KS) or Anderson-Darling (AD) assessments. These assessments provide primary 1° values for the TVT (as shown in FIG. 18), PVP (as shown in FIG. 19) and AVA (as shown in FIG. 20) component distributions. In this example, the primary 1° value graphs (FIGS. 18 to 20) are generated by the evaluation module 308 using a two sample AD assessment, with the primary 1° value for a given pairwise comparison being equal to the test statistic; whereby the horizontal line in the graphs denoting statistical significance at a p=0.05 level.

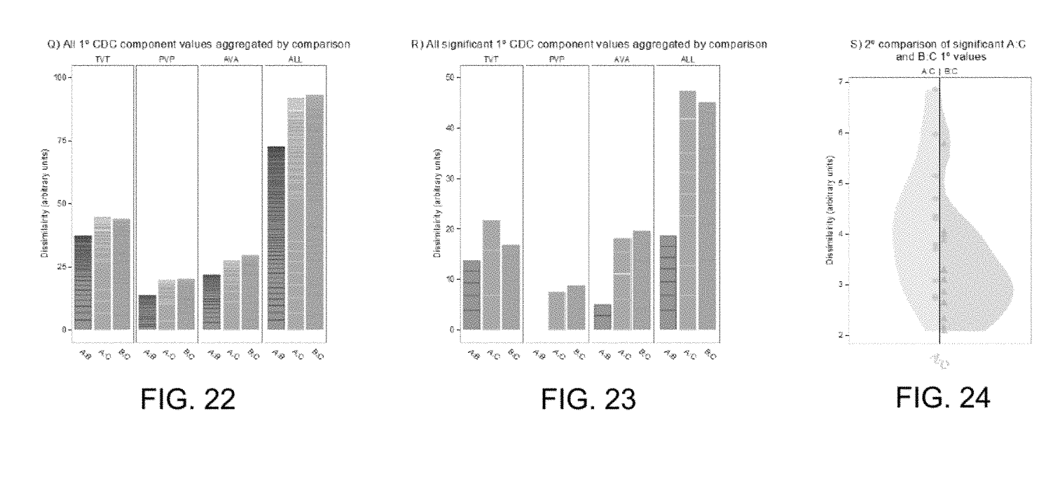

As shown, the evaluation module 308 is able to provide a comprehensive comparison between samples. In this example, 15 TVT, 5 PVP, and 12 AVA significant primary 1° values are determined and a number of differences are uniquely captured by a single component distribution. In cases of unequal metaphase numbers or chromosome complements, the relevant primary 1° values can be weighted as appropriate. In this example, primary 1° component values can be logically aggregated by the evaluation module 308 to determine the chromosomes with the greatest telomere differences between samples, as shown in FIG. 21. In further cases, the evaluation module 308 can quantify the total differences in telomeres between samples, as shown in FIGS. 22 and 23.

In further cases, the evaluation module 308 can provide secondary 2° comparisons of primary 1° value distributions, and similar higher order evaluations. As an example, while the aggregated primary 1° sample A to sample C (A:C) comparison values and sample B to sample C (B:C) comparison values all reach a similar total, as shown in FIG. 23, the value distributions, as shown in FIG. 24, can themselves be further compared. For example, the evaluation module 308 can determine the significance of the difference between the A:C and B:C comparisons; in this example, having a p-value of 0.0337 under the AD assessment. In this example, the distribution of the A:C primary 1° values and the B:C primary 1° values have been analyzed by the evaluation module 308 using the AD assessment. The evaluation module 308 determined that the values are significantly different, despite the values having essentially the same total value and would appear similar using conventional, less detailed analysis. The evaluation module 308 determines the specified p-value to be 0.0337.

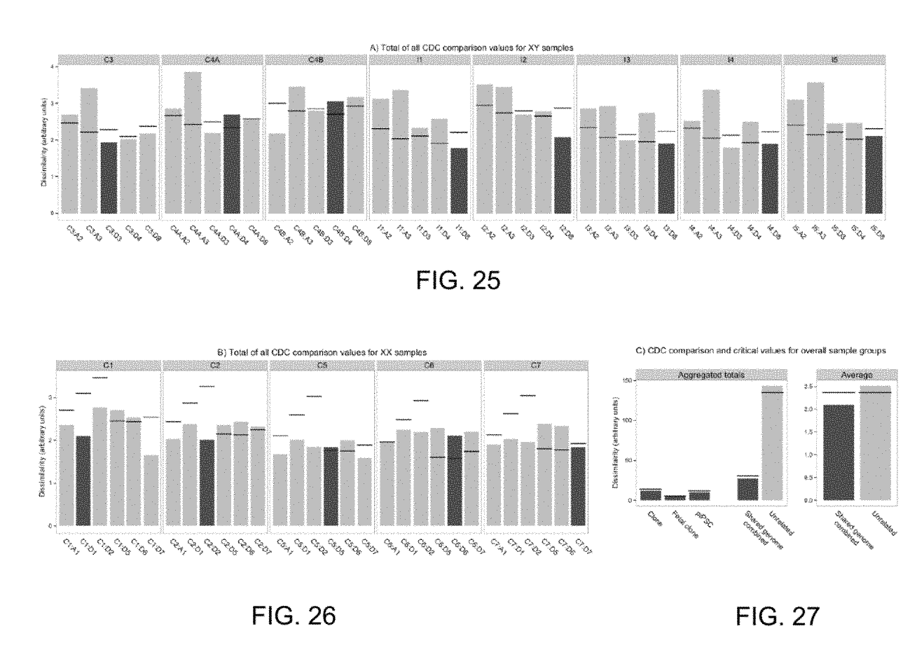

FIGS. 25 to 27 are an example illustrating an embodiment where the evaluation module 308 performs CDC assessments of telomere lengths between samples and sample types. Individual columns represent pairwise same sex comparisons in FIGS. 25 and 26, or comparison types in FIG. 27. Larger values represent a greater degree of dissimilarity, same genome comparisons are shown as darker and non-shared genome comparisons are shown as lighter. Where present black bars denote the threshold for significance at the p=0.05 level. Initial pairwise comparison analyses were completed between male samples in FIG. 25 and female samples in FIG. 26 before aggregation into sample groups shown in FIG. 27. The ratio version of CDC analysis with Kolmogorov-Smirnov tests was employed for comparison and weighting of sex chromosome values prior to aggregation.

In further embodiments, the system and method described herein may employ other multi-component datasets; for example, protein expression levels at different steps of a pathway, RNA expression data, population and behaviour datasets, or the like.

In the embodiments described herein, there is provided a technological solution to problems related to the measurement of telomere length. The fundamentally sizable, multifaceted and linked nature of the datasets to be analyzed necessitates the technological approach described herein. Particularly, the measurement data is comprised of large sets of different types of values, determined via the measurement technique, with various relationship considerations between them. The embodiments described herein provide a technological approach to arrive at a comprehensive and integrative analysis that captures detail from each individual measurement in the dataset and considers the relationships between such measurements. Accordingly, the system embodiments described herein require a technological approach in order to, for example: maintain the original dataset in full so that it can be referenced throughout analysis; subsect relevant details and values from the full dataset as necessary for the analysis; propagate relevant component distributions from the subsected values and information so that both individual measurements and their relationships can be analyzed; complete pairwise analysis between the component distributions; and integrate and aggregate the results of the pairwise analysis for higher order analysis. In some cases, the above analysis may be required to be further iterated upon. As such, the combination of sizable and richly detailed datasets, value propagations, non-trivial testing determinations, and nested iterative conditions reasonably and functionally necessitate that an implementation of the embodiments described herein incorporate technological means.

Although the invention has been described with reference to certain specific embodiments, various other aspects, advantages and modifications thereof will be apparent to those skilled in the art without departing from the spirit and scope of the invention as outlined in the claims appended hereto. The entire disclosures of all references recited above are incorporated herein by reference.

1. A computer-implemented method for patterning analysis of a telomere length dataset executed on a processing unit, the processing unit comprising one or more processors, the method comprising:
capturing a dataset of telomere length using a computer-assisted measurement technique;
generating component distribution datasets via analysis of the telomere length dataset;
generating datasets of primary values by performing pairwise assessments of levels of similarity between component distribution datasets;
generating datasets of higher order values comprising at least one higher order assessment of primary value datasets; and
outputting the datasets of primary values and the datasets of higher order values.

2. The method of claim 1, further comprising assigning a measure of significance to the primary values, the at least one higher order comparison values, or both.

3. The method of claim 2, wherein the primary values, the at least one higher order comparison values, or both, are weighted based on the assigned significance.

4. The method of claim 1, wherein the measurement technique is quantitative fluorescence in situ hybridization (FISH) on metaphase preparations (mqFISH).

5. The method of claim 4, wherein the measurement technique includes at least ten metaphases per sample.

6. The method of claim 1, wherein the measurement technique is normalized with a centromere reference probe by averaging two centromere measurements in each metaphase to normalize the raw telomere measurements of that metaphase.

7. The method of claim 1, wherein the measurement technique is normalized for each telomere length measurement by converting the measurements into a proportion of the sum of all telomere lengths considered in each given metaphase and component distribution.

8. The method of claim 1, wherein the measurement technique is normalized for each telomere length measurement by normalizing to the mean telomere length value per metaphase.

9. The method of claim 1, wherein each of the component distributions comprise one of a telomere versus telomere (TVT) distribution, a pair versus pair (PVP) distribution, and an arm versus arm (AVA) distribution.

10. The method of claim 1, wherein the primary values are determined by a non-parametric Anderson-Darling (AD) assessment.

11. The method of claim 1, wherein the primary values are determined by a Kolmogorov-Smirnov (KS) assessment.

12. The method of claim 1, wherein the higher order assessment is selected from a group consisting of: determining a full assessment of similarity between the primary values; determining a chromosome with the most telomere dissimilarity using the primary values; and determining a telomere with the most dissimilarity using the primary values.

13. A system for patterning analysis of a telomere length dataset, the system comprising one or more processors and a data storage device, the one or more processors configured to execute, or direct to be executed:
a measurement module for capturing a dataset of telomere length using a measurement technique from an input device;
a component module for generating component distribution datasets via analysis of the telomere length dataset;
an evaluation module for generating datasets of primary values by performing pairwise assessments of levels of similarity between component distribution datasets, and for generating datasets of higher order values comprising at least one higher order assessment of primary value datasets; and
an output module for outputting the datasets of primary values and the datasets of higher order values.

14. The system of claim 13, wherein the evaluation module assigns a measure of significance to the primary values, the at least one higher order comparison values, or both.

15. The system of claim 14, wherein the evaluation module weighs the primary values, the at least one higher order comparison values, or both, based on the assigned significance.

16. The system of claim 13, wherein the measurement technique is quantitative fluorescence in situ hybridization (FISH) on metaphase preparations (mqFISH).

17. The system of claim 13, wherein each of the component distributions comprise one of a telomere versus telomere (TVT) distribution, a pair versus pair (PVP) distribution, and an arm versus arm (AVA) distribution.

18. The system of claim 13, wherein the primary values are determined by a non-parametric Anderson-Darling (AD) assessment.

19. The system of claim 13, wherein the primary values are determined by a Kolmogorov-Smirnov (KS) assessment.

20. The system of claim 13, wherein the higher order assessment is selected from a group consisting of: determining a full comparison between the primary values; determining a chromosome with the most telomere dissimilarity using the primary values; and determining a telomere with the most dissimilarity using the primary values.
